(19) United States
(12) Patent Application Publication
(10) Pub. No.: US 2012/0093860 A1
(43) Pub. Date: Apr. 19, 2012

(54) INFLUENZA VACCINES WITH INCREASED AMOUNTS OF H3 ANTIGEN

(75) Inventors: Klaus Stohr, Cambridge, MA (US); Theodore Tsai, Cambridge, MA (US)

(73) Assignee: NOVARTIS AG, Basel (CH)

(21) Appl. No.: 13/148,947

(22) PCT Filed: Feb. 10, 2010
(86) PCT No.: PCT/IB2010/000305

§ 371 Date: Dec. 12, 2011

Related U.S. Application Data
(60) Provisional application No. 61/207,368, filed on Feb. 10, 2009.

(51) Int. Cl. A61K 39/145; A61P 31/16; A61P 37/04

(52) U.S. Cl. 424/210.1

(57) ABSTRACT

An influenza vaccine includes an increased amount of H3N2 antigen relative to the normal dose. In a typical embodiment, the vaccine includes hemagglutinins from an A/H1N1 strain, an A/H3N2 strain, and a B strain, wherein (i) the weight ratio of H3N2:H1N1 hemagglutinin is greater than 1 and (ii) the weight ratio of H3N2:B hemagglutinin is greater than 1. In such a vaccine the weight ratio of H1N1:B hemagglutinin will normally be 1. For example, a vaccine may contain hemagglutinin at 15 μg for A/H1N1, 30 μg for A/H3N2 and 15 μg for B.

This patent application claims priority from United States provisional patent application 61/207,368, filed 10th Feb. 2009, the complete contents of which are incorporated herein by reference.

### TECHNICAL FIELD

This invention is in the field of vaccines for protecting against influenza virus infection, and in particular vaccines that include increased amounts of influenza A virus H3 antigen.

### BACKGROUND ART

Current seasonal influenza vaccines typically include antigens from two influenza A strains (H1N1 and H3N2) and one influenza B strain.

Reference 1 reported that the 2005 southern hemisphere formulation of the inactivated split-virion influenza vaccine VAXIGRIP™ contained a lower concentration of haemagglutinin than desired. Although the vaccine included the standard 15 μg dose for the A/H1N1 and B strains, it contained only 9 μg for the A/H3N2 strain. Even so, the authors found that the vaccine met the immunogenicity criteria for annual licensure of influenza vaccines in Europe.

It is an object of the invention to provide further and improved formulations of seasonal influenza vaccines.

### DISCLOSURE OF THE INVENTION

In contrast to reference 1, according to the invention an influenza vaccine includes an increased amount of H3N2 antigen relative to the normal dose.

Thus the invention provides an influenza virus vaccine including A/H3N2 hemagglutinin, wherein the concentration of A/H3N2 hemagglutinin is greater than 16 μg per human dose.

The invention also provides an influenza virus vaccine including A/H3N2 hemagglutinin, wherein the concentration of A/H3N2 hemagglutinin is greater than 32 μg/mL.

The invention also provides an influenza virus vaccine including an A/H1N1 hemagglutinin and a A/H3N2 hemagglutinin, wherein the weight ratio of H3N2:H1N1 hemagglutinin is greater than I.

The invention also provides an influenza virus vaccine including an A/H1N1 hemagglutinin and a B hemagglutinin, wherein the weight ratio of H3N2:B hemagglutinin is greater than 1.

In a typical embodiment, the vaccine includes hemagglutinins from an A/H1N1 strain, an A/H3N2 strain, and a B strain, wherein (i) the weight ratio of H3N2:H1N1 hemagglutinin is greater than 1 and (ii) the weight ratio of H3N2:B hemagglutinin is greater than 1. In such a vaccine the weight ratio of H1N1:B hemagglutinin will normally be 1.

Vaccine Preparation

Various forms of influenza virus vaccine are currently available, and vaccines are generally based either on live virus or on inactivated virus. Inactivated vaccines may be based on whole virions, split virions, or on purified surface antigens. Influenza antigens can also be presented in the form of virosomes. The invention can be used with any of these types of vaccine, but will typically be used with inactivated vaccines.

Where an inactivated virus is used, the vaccine may comprise whole virion, split virion, or purified surface antigens (including hemagglutinin and, usually, also including neuraminidase). Chemical means for inactivating a virus include treatment with an effective amount of one or more of the following agents: detergents, formaldehyde, β-propiolactone, methylene blue, psoralen, carboxyfullerene (C60), binary ethylamine, acetyl ethyleneimine, or combinations thereof. Non-chemical methods of viral inactivation are known in the art, such as for example UV light or gamma irradiation.

Virions can be harvested from virus-containing fluids by various methods. For example, a purification process may involve zonal centrifugation using a linear sucrose gradient solution that includes detergent to disrupt the virions. Antigens may then be purified, after optional dilution, by diafiltration.

Split virions are obtained by treating purified virions with detergents (e.g. ethyl ether, polysorbate 80, deoxycholate, tri-N-butyl phosphate, Triton X-100, Triton N101, cetyltrimethylammonium bromide, Tergitol NP9, etc.) to produce subvirion preparations, including the ‘Tween-ether’ splitting process. Methods of splitting influenza viruses are well known in the art e.g. see refs. 2-7, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g. cationic) surfactants e.g. alkylglycosides, alkylthioglycosides, acyl sugars, sulphobetaines, betains, polyoxyethylenealkylethers, N,N-dialkyl-Glucamides, Hecameg, alkylphenoxy-polyethoxyethanols, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonylphenoxy polyoxyethanols (e.g. the Triton surfactants, such as Triton X-100 or Triton N101), polyoxyethylene sorbitan esters (the Tween surfactants), polyoxyethylene ethers, polyoxyethlene esters, etc. One useful splitting procedure uses the consecutive effects of sodium deoxycholate and formaldehyde, and splitting can take place during initial virion purification (e.g. in a sucrose density gradient solution). Thus a splitting process can involve clarification of the virion-containing material (to remove non-virion material), concentration of the harvested virions (e.g. using an adsorption method, such as CaHPO₄ adsorption), separation of whole virions from non-virion material, splitting of virions using a splitting agent in a density gradient centrifugation step (e.g. using a sucrose gradient that contains a splitting agent such as sodium deoxycholate), and then filtration (e.g. ultrafiltration) to remove undesired materials. Split virions can usefully be resuspended in sodium phosphate-buffered isotonic sodium chloride solution. The BEGRIVAC™, FLUARIXυ, FLUZONE™ and FLUSHIELD™ products are split vaccines. Purified surface antigen vaccines comprise the influenza surface antigens haemagglutinin and, typically, also neuraminidase. Processes for preparing these proteins in purified form are well known in the art. The FLUVIRIN™, AGRIPPAL™ and INFLUVAC™ products are examples.

Another form of inactivated influenza antigen is the virosome [8] (nucleic acid free viral-like liposomal particles), as in the INFLEXAL V™ and INVAVAC™ products. Virosomes can be prepared by solubilization of influenza virus with a detergent followed by removal of the nucleocapsid and reconstitution of the membrane containing the viral glycoproteins. An alternative method for preparing virosomes involves adding viral membrane glycoproteins to excess amounts of phospholipids, to give liposomes with viral proteins in their membrane.

Strains used with the invention may have a natural HA as found in a wild-type virus, or a modified HA. For instance, it is known to modify HA to remove determinants (e.g. hyper-basic regions around the HA1/HA2 cleavage site) that cause a virus to be highly pathogenic in avian species. Hemagglutinins of influenza B viruses used with the invention preferably have Asn at amino acid 197, providing a glycosylation site [9].

An influenza virus used with the invention may be a reassortant strain, and may have been obtained by reverse genetics techniques. Reverse genetics techniques [e.g. 10-14] allow influenza viruses with desired genome segments to be prepared in vitro using plasmids or other artificial vectors. Typically, it involves expressing (a) DNA molecules that encode desired viral RNA molecules e.g. from poll promoters or bacteriophage RNA polymerase promoters, and (b) DNA molecules that encode viral proteins e.g. from poII promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA can be used [15-17], and these methods will also involve the use of plasmids to express all or some (e.g. just the PBI, PB2, PA and NP proteins) of the viral proteins, with up to 12 plasmids being used in some methods.

To reduce the number of plasmids needed, one approach [18] combines a plurality of RNA polymerase I transcription cassettes (for viral RNA synthesis) on the same plasmid (e.g. sequences encoding 1, 2, 3, 4, 5, 6, 7 or all 8 influenza A vRNA segments), and a plurality of protein-coding regions with RNA polymerase II promoters on another plasmid (e.g. sequences encoding 1, 2, 3, 4, 5, 6, 7 or all 8 influenza A mRNA transcripts). Preferred aspects of the reference 18 method involve: (a) PB1, PB2 and PA mRNA-encoding regions on a single plasmid; and (b) all 8 vRNA-encoding segments on a single plasmid. Including the NA and HA segments on one plasmid and the six other segments on another plasmid can also facilitate matters.

As an alternative to using poll promoters to encode the viral RNA segments, it is possible to use bacteriophage polymerase promoters [19]. For instance, promoters for the SP6, T3 or T7 polymerases can conveniently be used. Because of the species-specificity of poll promoters, bacteriophage polymerase promoters can be more convenient for many cell types (e.g. MDCK), although a cell must also be transfected with a plasmid encoding the exogenous polymerase enzyme.

In other techniques it is possible to use dual polI and polII promoters to simultaneously code for the viral RNAs and for expressible mRNAs from a single template [20,21].

Thus an influenza A virus may include one or more RNA segments from a A/PR/8/34 virus (typically 6 segments from A/PR/8/34, with the HA and N segments being from a vaccine strain, i.e. a 6:2 reassortant). It may also include one or more RNA segments from a A/WSN/33 virus, or from any other virus strain useful for generating reassortant viruses for vaccine preparation. An influenza A virus may include fewer than 6 (i.e. 0, 1, 2, 3, 4 or 5) viral segments from an AA/6/60 influenza virus (A/Ann Arbor/6/60). An influenza B virus may include fewer than 6 (i.e. 0, 1, 2, 3, 4 or 5) viral segments from an AA/1/66 influenza virus (B/Ann Arbor/1/66). Typically, the invention protects against a strain that is capable of human-to-human transmission, and so the strain's genome will usually include at least one RNA segment that originated in a mammalian (e.g. in a human) influenza virus. It may include NS segment that originated in an avian influenza virus.

Strains whose antigens can be included in the compositions may be resistant to antiviral therapy (e.g. resistant to oseltamivir [22] and/or zanamivir), including resistant pandemic strains [23].

Particularly useful strains are those that have not been passaged through eggs at any stage between isolation from a patient and replication in a cell culture system, inclusive. MDCK cells can be used exclusively of for all steps from isolation to virus replication.

In some embodiments, strains used with the invention have hemagglutinin with a binding preference for oligosaccharides with a Sia(α2,6)Gal terminal disaccharide compared to oligosaccharides with a Sia(α2,3)Gal terminal disaccharide. Human influenza viruses bind to receptor oligosaccharides having a Sia(α2,6)Gal terminal disaccharide (sialic acid linked α-2,6 to galactose), but eggs and Vero cells have receptor oligosaccharides with a Sia(α2,3)Gal terminal disaccharide. Growth of human influenza viruses in cells such as MDCK provides selection pressure on hemagglutinin to maintain the native Sia(α2,6)Gal binding, unlike egg passaging.

To determine if a virus has a binding preference for oligosaccharides with a Sia(α2,6)Gal terminal disaccharide compared to oligosaccharides with a Sia(a2,3)Gal terminal disaccharide, various assays can be used. For instance, reference 24 describes a solid-phase enzyme-linked assay for influenza virus receptor-binding activity which gives sensitive and quantitative measurements of affinity constants. Reference 25 used a solid-phase assay in which binding of viruses to two different sialylglycoproteins was assessed (ovomucoid, with Sia(α2,3)Gal determinants; and pig α₂-macroglobulin, which Sia(α2,6)Gal determinants), and also describes an assay in which the binding of virus was assessed against two receptor analogs: free sialic acid (Neu5Ac) and 3′-sialyllactose (Neu5Acα2-3Galβ1-4Glc). Reference 26 reports an assay using a glycan array which was able to clearly differentiate receptor preferences for α2,3 or α2,6 linkages. Reference 27 reports an assay based on agglutination of human erythrocytes enzymatically modified to contain either Sia(α2,6)Gal or Sia(α2,3)Gal. Depending on the type of assay, it may be performed directly with the virus itself, or can be performed indirectly with hemagglutinin purified from the virus.

In some embodiments influenza strains used with the invention have glycoproteins (including hemagglutinin) with a different glycosylation pattern from egg-derived viruses. Thus the glycoproteins will include glycoforms that are not seen in chicken eggs.

Hemagglutinin Dosing

Hemagglutinin (HA) is the main immunogen in current inactivated influenza vaccines, and vaccine doses are standardised by reference to HA levels, typically measured by SRID. Existing vaccines typically contain about 15 μg of HA per strain, although lower doses can be used e.g. for children, or in pandemic situations, or when using an adjuvant, or by accident. Fractional doses such as ½ (i.e. 7.5 μg HA per strain), ¼ and ⅛have been used, as have higher doses (e.g. 3× or 9× doses [28,29]).

The standard influenza vaccine volume is 0.5 ml, and so the standard HA concentration for each strain in a vaccine is 30 μg/ml. According to the invention, however, an influenza vaccine includes an increased amount of H3N2 antigen. Thus the concentration of HA from A/H3N2 may be greater than 32 μg/mL. For example, it may be greater than 35 μg/ml, greater than 40 μg/ml, greater than 45 μg/ml, greater than 50 μg/ml, greater than 55 μg/ml, greater than 60 μg/ml, greater than 65 μg/ml, greater than 70 μg/ml, etc. The concentration will not normally exceed 300 μg/ml i.e. it is in the range from 32-300 μg/ml. In one useful embodiment, the concentration of A/H3N2 hemagglutinin is about 60 μg/ml i.e. twice the standard dose. Thus a useful vaccine can comprise 60 μg/ml A/H3N2 hemagglutinin.

With a 0.5 ml volume, therefore, the amount of A/H3N2 HA in a single dose will be greater than 16 μg e.g. greater than 17.5 mg, greater than 20 μg, greater than 25 μg, greater than 25μg, greater than 27.5 μg, greater than 30 μg, greater than 32.5 μg, greater than 35 μg, etc. but the amount will not normally exceed 150 μg. In one useful embodiment, a dose comprises 30 μg A/H3N2 hemagglutinin.

For other strains in the vaccine (e.g. an A/H1N1 strain and an influenza B virus strain) the amount of HA per dose can be in the range 0.1 and 150 μg per strain, preferably between 0.1 and 50 μg e.g. 0.1-20 μg, 0.1-1.5 μg, 0.1-10 μg, 0.1-7.5μg, 0.5-5 μg, etc. Particular doses include e.g. about 45, about 30, about 15, about 10, about 7.5, about 5, about 3.8, about 1.9, about 1.5, etc. μg per strain.

Ideally, however, the amount of A/H3N2 hemagglutinin is higher than both the amount of A/H1N1 HA and the amount of influenza B virus HA. Thus the weight ratio of H3N2:H1N1 hemagglutinin is greater than 1, and preferably at least 1.25 e.g. at least 1.5, 1.75. 2.0, 2.25, 2.5, 2.75, 3.0, etc. Similarly, the weight ratio of H3N2 hemagglutinin to influenza B virus hemagglutinin is greater than 1, and preferably at least 1.25 e.g. at least 1.5, 1.75, 2.0, 2.25, 2.5, 2.75, 3.0, etc. The weight ratio of A/H1N1:B hemagglutinin will normally be about 1. For example, in a vaccine containing hemagglutinin at 15 μg for A/H1N1, 30 μg for A/H3N2 and 15 μg for B, ratio of A/H3N2 to A/H1N1 is 2:1 and the ratio of A/H3N2 to B is also 2:1, with the ratio of A/H1N1 to B being 1.

For a vaccine including hemagglutinin from multiple human influenza virus strains (e.g. from A/H1N1, A/H3N2 and B) the amount of A/H3N2 hemagglutinin is preferably higher than the average (mean) amount of hemagglutinin per strain. The amount of A/H3N2 hemagglutinin may be at least 1.25× the mean e.g. at least 1.5, 1.75, 2.0, 2.25, 2.5, etc. For example, in a vaccine containing hemagglutinin at 15 μg for A/H1N1, 30 μg for A/H3N2 and 15 μg for B, the mean amount of HA per strain is 20 μg and so the amount of A/H3N2 is 1.5× the mean.

The amount of A/H3N2 hemagglutinin may be equal to the combined amount of A/H1N1 hemagglutinin and B hemagglutinin. For example, in a vaccine containing hemagglutinin at 15 μg for A/H1N1 and 15 μg for B, the amount of A/H3N2 hemagglutinin may be 30 μg.

If a vaccine composition includes hemagglutinin from more than one influenza B virus strain (for example, reference 30 discloses 4-valent vaccines with HA from two A strains and two B strains) the above statements apply to one of those strains. If a vaccine includes HA from both a B/Victoria/2/87-like strain and a B/Yamagata/16/88-like strain, the above statements apply to either the B/Victoria/2/87-like strain or the B/Yamagata/16/88-like strain. These two lineages emerged in the late 1980s and have HAs which can be antigenically and/or genetically distinguished from each other [31]. B/Yamagata/16/88-like strains often (but not always) have HA proteins with deletions at amino acid residue 164, numbered relative to the ‘Lee40’ HA sequence [32].

A vaccine composition may include, in addition to a seasonal strain, hemagglutinin from a pandemic-associated strain. For example, reference 30 discloses vaccines including A/H1N1, A/H3N2, A/H5N1 and B antigens. Influenza A virus currently displays sixteen HA subtypes: H1, H2, H3, H4, H5, H6, H7, F18, H9, H10, H11, H12, H13, H14, H15 and H16. Characteristics of a pandemic-associated influenza strain are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the vaccine recipient and the general human population are immunologically naïve to the strain's hemagglutinin; (b) it is capable of being transmitted horizontally in the human population; and (c) it is pathogenic to humans. A pandemic-associated influenza virus strain for use with the invention will typically have a H2, H5, H7 or H9 subtype e.g. H5N1, H5N3, H9N2, H2N2, H7N1 or H7N7. H5N1 strains are preferred. A pandemic-associated strain can also have a H1 subtype (e.g. H1N1); for example, its HA can be immunologically cross-reactive with the A/California/04/09 strain.

The amount of A/H3N2 HA will, as described above, be greater than 1.5× the amount of A/H5N1 HA e.g. 2×, 2.5×, 3×, 3.5×, 4×, etc.

Typically, however, the vaccine will include antigen from only three influenza virus strains, namely a H1N1 influenza virus A strain, a H3N2 influenza virus A strain, and an influenza B virus strain. The influenza B virus strain will usually be a B/Victoria/2/87-like or a B/Yamagata/16/88-like strain. The HA weight ratio in such trivalent vaccines is preferably 1:2:1 (A/H1N1:A/H3N2:B).

In some embodiments the H3 strain is cross-reactive with A/Moscow/10/99. In other embodiments it is cross-reactive with A/Fujian/411/2002.

Live Vaccines

As mentioned above, the invention will usually be used with inactivated vaccines. In some embodiments, however, it can be used with live vaccines. Rather than being standardized around HA content, live vaccines dosing is measured by median tissue culture infectious dose (TCID₅₀). A TCID₅₀ of between 10⁶ and 10⁸ (preferably between 10^{6.5}-10^{7.5}) per strain is typical and so, according to the invention, the H3N2 dose is higher than normal, with the above-mentioned ratios etc. being applied to the H3N2 TCID₅₀ e.g. with a TCID₅₀ being 2×10⁷ for H3N2, but 1×10⁷ for other strains.

The influenza virus may be attenuated.

The influenza virus may be temperature-sensitive.

The influenza virus may be cold-adapted.

Cell lines

Manufacture of vaccines for use with the invention can use SPF eggs as the substrate for viral growth, wherein virus is harvested from infected allantoic fluids of hens' eggs. Instead, however, cell lines which support influenza virus replication may be used. The cell line will typically be of mammalian origin. Suitable mammalian cells of origin include, but are not limited to, hamster, cattle, primate (including humans and monkeys) and dog cells, although the use of primate cells is not preferred. Various cell types may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line [33-35]. Suitable dog cells are e.g. kidney cells, as in the CLDK and MDCK cell lines.

Thus suitable cell lines include, but are not limited to: MDCK; CHO; CLDK; HKCC; 293T; BHK; Vero; MRC-5; PER.C6 [36]; FRhL2; WI-38; etc. Suitable cell lines are widely available e.g. from the American Type Cell Culture (ATCC) collection [37], from the Coriell Cell Repositories [38], or from the European Collection of Cell Cultures (ECACC). For example, the ATCC supplies various different Vero cells under catalog numbers CCL-81, CCL-81.2, CRL-1586 and CRL-1587, and it supplies MDCK cells under catalog number CCL-34. PER.C6 is available from the ECACC under deposit number 96022940.

The most preferred cell lines are those with mammalian-type glycosylation. As a less-preferred alternative to mammalian cell lines, virus can be grown on avian cell lines [e.g. refs. 39-41], including cell lines derived from ducks (e.g. duck retina) or hens. Examples of avian cell lines include avian embryonic stem cells [39,42] and duck retina cells [40]. Suitable avian embryonic stem cells, include the EBx cell line derived from chicken embryonic stem cells, EB45, EB 14, and EB14-074 [43]. Chicken embryo fibroblasts (CEF) may also be used. Rather than using avian cells, however, the use of mammalian cells means that vaccines can be free from avian DNA and egg proteins (such as ovalbumin and ovomucoid), thereby reducing allergenicity.

The most preferred cell lines for growing influenza viruses are MDCK cell lines [44-47], derived from Madin Darby canine kidney. The original MDCK cell line is available from the ATCC as CCL-34, but derivatives of this cell line and other MDCK cell lines may also be used. For instance, reference 44 discloses a MDCK cell line that was adapted for growth in suspension culture (‘MDCK 33016’, deposited as DSM ACC 2219). Similarly, reference 48 discloses a MDCK-derived cell line that grows in suspension in serum-free culture (‘B-702’, deposited as FERM BP-7449). Reference 49 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (PTA-6503). Reference 50 discloses MDCK cell lines with high susceptibility to infection, including ‘MDCK.5F1’ cells (ATCC CRL-12042). Any of these MDCK cell lines can be used.

Virus may be grown on cells in adherent culture or in suspension. Microcarrier cultures can also be used. In some embodiments, the cells may thus be adapted for growth in suspension.

Cell lines are preferably grown in serum-free culture media and/or protein free media. A medium is referred to as a serum-free medium in the context of the present invention in which there are no additives from serum of human or animal origin. The cells growing in such cultures naturally contain proteins themselves, but a protein-free medium is understood to mean one in which multiplication of the cells (e.g. prior to infection) occurs with exclusion of proteins, growth factors, other protein additives and non-serum proteins, but can optionally include proteins such as trypsin or other proteases that may be necessary for viral growth.

Cell lines supporting influenza virus replication are preferably grown below 37° C. [51] (e.g. 30-36° C., or at about 30° C., 31° C., 32° C., 33° C., 34° C., 35° C., 36° C.) during viral replication.

Methods for propagating influenza virus in cultured cells generally includes the steps of inoculating a culture of cells with an inoculum of the strain to be grown, cultivating the infected cells for a desired time period for virus propagation, such as for example as determined by virus titer or antigen expression (e.g. between 24 and 168 hours after inoculation) and collecting the propagated virus. The cultured cells are inoculated with a virus (measured by PFU or TCID₅₀) to cell ratio of 1:500 to 1:1, preferably 1:100 to 1:5, more preferably 1:50 to 1:10. The virus is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g. monolayers) may be removed either by freeze-thawing or by enzymatic action to increase the viral content of the harvested culture supernatants. The harvested fluids are then either inactivated or stored frozen. Cultured cells may be infected at a multiplicity of infection (“m.o.i.”) of about 0.0001 to 10, preferably 0.002 to 5, more preferably to 0.001 to 2. Still more preferably, the cells are infected at a m.o.i of about 0.01. Infected cells may be harvested 30 to 60 hours post infection. Preferably, the cells are harvested 34 to 48 hours post infection. Still more preferably, the cells are harvested 38 to 40 hours post infection. Proteases (typically trypsin) are generally added during cell culture to allow viral release, and the proteases can be added at any suitable stage during the culture e.g. before inoculation, at the same time as inoculation, or after inoculation [51].

In useful embodiments, particularly with MDCK cells, a cell line is not passaged from the master working cell bank beyond 40 population-doubling levels.

The viral inoculum and the viral culture are preferably free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses [52]. Absence of herpes simplex viruses is particularly preferred.

Host Cell DNA

Where virus has been grown on a cell line then it is standard practice to minimize the amount of residual cell line DNA in the final vaccine, in order to minimize any oncogenic activity of the DNA.

Thus a vaccine composition prepared according to the invention preferably contains less than 10 ng (preferably less than 1 ng, and more preferably less than 100 μg) of residual host cell DNA per dose, although trace amounts of host cell DNA may be present.

Vaccines containing <10 ng (e.g. <1 ng, <100 μg) host cell DNA per 15 μg of haemagglutinin are preferred, as are vaccines containing <10 ng (e.g. <1 ng, <100 μg) host cell DNA per 0.25 ml volume. Vaccines containing <10 ng (e.g. <1 ng, <100 μg) host cell DNA per 50 μg of haemagglutinin are more preferred, as are vaccines containing <10 ng (e.g. <1 ng, <100 μg) host cell DNA per 0.5 ml volume.

It is preferred that the average length of any residual host cell DNA is less than 500 bp e.g. less than 400 bp, less than 300 bp, less than 200 bp, less than 100 bp, etc.

Contaminating DNA can be removed during vaccine preparation using standard purification procedures e.g. chromatography, etc. Removal of residual host cell DNA can be enhanced by nuclease treatment e.g. by using a DNase. A convenient method for reducing host cell DNA contamination is disclosed in references 53 & 54, involving a two-step treatment, first using a DNase (e.g. Benzonase), which may be used during viral growth, and then a cationic detergent (e.g. CTAB), which may be used during virion disruption. Removal by (3-propiolactone treatment can also be used.

Measurement of residual host cell DNA is now a routine regulatory requirement for biologicals and is within the normal capabilities of the skilled person. The assay used to measure DNA will typically be a validated assay [55,56]. The performance characteristics of a validated assay can be described in mathematical and quantifiable terms, and its possible sources of error will have been identified. The assay will generally have been tested for characteristics such as accuracy, precision, specificity. Once an assay has been calibrated (e.g against known standard quantities of host cell DNA) and tested then quantitative DNA measurements can be routinely performed. Three main techniques for DNA quantification can be used: hybridization methods, such as Southern blots or slot blots [57]; immunoassay methods, such as the Threshold™ System [58]; and quantitative PCR [59]. These methods are all familiar to the skilled person, although the precise characteristics of each method may depend on the host cell in question e.g. the choice of probes for hybridization, the choice of primers and/or probes for amplification, etc. The Threshold™ system from Molecular Devices is a quantitative assay for picogram levels of total DNA, and has been used for monitoring levels of contaminating DNA in biopharmaceuticals [58]. A typical assay involves non-sequence-specific formation of a reaction complex between a biotinylated ssDNA binding protein, a urease-conjugated anti-ssDNA antibody, and DNA. All assay components are included in the complete Total DNA Assay Kit available from the manufacturer. Various commercial manufacturers offer quantitative PCR assays for detecting residual host cell DNA e.g. AppTec™ Laboratory Services, BioReliance™, Althea Technologies, etc. A comparison of a chemiluminescent hybridisation assay and the total DNA Threshold™ system for measuring host cell DNA contamination of a human viral vaccine can be found in reference 60.

Pharmaceutical Compositions

Vaccines for use with the invention usually include components in addition to the influenza antigens e.g. they typically include one or more pharmaceutical carrier(s) and/or excipient(s). A thorough discussion of such components is available in reference 61. In many embodiments adjuvants may also be included, particularly for vaccines administered by intramuscular injection.

Compositions will generally be in aqueous form at the point of administration.

A composition may include preservatives such as thiomersal or 2-phenoxyethanol. It is preferred that the vaccine should be substantially free from (e.g. <10 μg/ml) mercurial material e.g. thiomersal-free [6,62]. Vaccines containing no mercury are more preferred, and a-tocopherol succinate can be included as an alternative to mercurial compounds [6]. Preservative-free vaccines are particularly preferred.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate, and/or magnesium chloride, etc. Where adjuvant is in a separate container from antigens, sodium chloride may be present in both containers.

Compositions may have an osmolality of between 200 mOsm/kg and 400 mOsm/kg, preferably between 240-360 mOsm/kg, maybe within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

Compositions of the invention may include detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent may be present only at trace amounts. Thus the vaccine may include less than 1 mg/ml of each of octoxynol-10 and polysorbate 80. Other residual components in trace amounts could be antibiotics (e.g. neomycin, kanamycin, polymyxin B). Where adjuvant is in a separate container from antigens, this detergent will usually be present in the antigen-containing container (e.g. antigen with polysorbate 80 and Octoxynol 10).

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Influenza vaccines are typically administered in a dosage volume of about 0.5 ml, although a half dose (i.e. about 0.25 ml) may also be administered, particularly to children. Lower doses (e.g. 0.1 ml or 0.2 ml) are also useful for administration routes such as intradermal injection.

Vaccines are preferably stored at between 2° C. and 8° C. They should not be frozen. They should ideally be kept out of direct light.

Vaccines may be supplied in any suitable container, either formulated ready for administration or as a kit of parts for extemporaneous mixing prior to administration e.g. as separate antigen and adjuvant components (as in the PREPANDRIX™ product). Suitable containers include vials, syringes (e.g. disposable syringes), nasal sprays, etc. These containers should be sterile. Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. The vial may include a single dose of vaccine, or it may include more than one dose (a ‘multidose’ vial) e.g. 10 doses. Preferred vials are made of colorless glass. A vial can have a cap (e.g. a Luer lock) adapted such that a syringe can be inserted into the cap. A vial may have a cap that permits aseptic removal of its contents, particularly for multidose vials. Where a component is packaged into a syringe, the syringe may have a needle attached to it. If a needle is not attached, a separate needle may be supplied with the syringe for assembly and use. Such a needle may be sheathed. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of a butyl rubber.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

A container may be packaged (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

Adjuvants

At the point of use, vaccines of the invention may advantageously include an adjuvant, which can function to enhance the immune responses (humoral and/or cellular) elicited in a patient who receives the composition. The presence of an oil-in-water emulsion adjuvant (particularly one comprising squalene) has been shown to enhance the strain cross-reactivity of immune responses for seasonal [63] and pandemic [64,65] influenza vaccines.

Oil-in-water emulsions for use with the invention typically include at least one oil and at least one surfactant, with the oil(s) and surfactant(s) being biodegradable (metabolisable) and biocompatible. The oil droplets in the emulsion are generally less than 5 μm in diameter, and may even have a sub-micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil, etc. In the grain group, corn oil is the most readily available, but the oil of other cereal grains such as wheat, oats, rye, rice, teff, triticale, etc. may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, can also be used. Fish oils, including squalene and squalane, are readily available from commercial sources or may be obtained by methods known in the art. Squalene is preferred.

Other useful oils are the tocopherols, which are advantageously included in vaccines for use in elderly patients (e.g. aged 60 years or older) because vitamin E has been reported to have a positive effect on the immune response in this patient group [66]. They also have antioxidant properties that may help to stabilize the emulsions [67]. Various tocopherols exist (α, β, γ, δ, ε or ξ but a is usually used. A preferred α-tocopherol is DL-α-tocopherol. α-tocopherol succinate is known to be compatible with influenza vaccines and to be a useful preservative as an alternative to mercurial compounds [6].

Mixtures of oils can be used e.g. squalene and a-tocopherol. An oil content in the range of 2-20% (by volume) is typical.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (1GEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. The most preferred surfactant for including in the emulsion is polysorbate 80 (polyoxyethylene sorbitan monooleate; Tween 80).

Mixtures of surfactants can be used e.g. Tween 80/Span 85 mixtures. A combination of a polyoxyethylene sorbitan ester and an octoxynol is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Squalene-containing oil-in-water emulsions are preferred, particularly those containing polysorbate 80. The weight ratio of squalene:polysorbate 80 may be between 1 and 10, for example between 2 and 9 e.g. about 2.2 or about 8.3. Specific oil-in-water emulsion adjuvants useful with the invention include, but are not limited to:
A submicron emulsion of squalene, polysorbate 80, and sorbitan trioleate. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [68-70], as described in more detail in Chapter 10 of ref. 71 and chapter 12 of ref. 72. The MF59 emulsion advantageously includes citrate ions e.g. 10 mM sodium citrate buffer.
A submicron emulsion of squalene, a tocopherol, and polysorbate 80. These emulsions may have from 2 to 10% squalene, from 2 to 10% tocopherol and from 0.3 to 3% polysorbate 80, and the weight ratio of squalene:tocopherol is preferably <1 (e.g. 0.90) as this can provide a more stable emulsion. Squalene and polysorbate 80 may be present at a volume ratio of about 5:2 or at a weight ratio of about 11:5. One such emulsion can be made by dissolving Tween 80 in PBS to give a 2% solution, then mixing 90 ml of this solution with a mixture of (5 g of DL-a-tocopherol and 5 ml squalene), then microfluidising the mixture. The resulting emulsion has submicron oil droplets e.g. with an average diameter of between 100 and 250nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [73].
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g an a-tocopherol succinate). The emulsion may include these three components at a mass ratio of about 75:11:10 (e.g. 750 μg/ml polysorbate 80, 110 μg/ml Triton X-100 and 100μg/ml a-tocopherol succinate), and these concentrations should include any contribution of these components from antigens. The emulsion may also include squalene. The emulsion may also include a 3d-MPL. The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [74] (0.05-1% Thr-MDP, 5% squalane, 2.5% Pluronic L121 and 0.2% polysorbate 80). It can also be used without the Thr-MDP, as in the “AF” adjuvant [75] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [76]. The emulsion may also include one or more of: alditol; a cryoprotective agent (e.g. a sugar, such as dodecylmaltoside and/or sucrose); and/or an alkylpolyglycoside. The emulsion may include a TLR4 agonist [77]. Such emulsions may be lyophilized.
An emulsion of squalene, poloxamer 105 and Abil-Care [78]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 79, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 80, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion in which a saponin (e.g. QuilA or QS21) and a sterol (e.g. a cholesterol) are associated as helical micelles [81].
An emulsion comprising a mineral oil, a non-ionic lipophilic ethoxylated fatty alcohol, and a non-ionic hydrophilic surfactant (e.g an ethoxylated fatty alcohol and/or polyoxyethylene-polyoxypropylene block copolymer) [82].
An emulsion comprising a mineral oil, a non-ionic hydrophilic ethoxylated fatty alcohol, and a non-ionic lipophilic surfactant (e.g an ethoxylated fatty alcohol and/or polyoxyethylene-polyoxypropylene block copolymer) [82].

The emulsions may be combined with antigen(s) during vaccine manufacture, or may be supplied as a separate component for mixing with a separate antigen-containing component extemporaneously, at the time of delivery (as in the PREPANDRIX™ product). Where these two components are liquids then the volume ratio of the two liquids for mixing can vary (e.g. between 5:1 and 1:5) but is generally about 1:1.

After the antigen and adjuvant have been mixed, haemagglutinin antigen will generally remain in aqueous solution but may distribute itself around the oil/water interface. In general, little if any haemagglutinin will enter the oil phase of the emulsion.

Preferred oil-in-water emulsion adjuvants comprise squalene. Such emulsions are already included in the FLUAD™ and PREPANDRIX™ products. In the FLUAD™ vaccine the total amount of HA is 45 μg (3×15 μg) and the total amount of squalene is 9.75 mg, in a dosage volume of 0.5 ml. In the PREPANDRIX™ vaccine the total amount of HA is 3.75 μg (monovalent) and the total amount of squalene is 10.68 mg, also in a dosage volume of 0.5 ml. Some embodiments of the invention use a lower amount of squalene per dose while still retaining an adjuvant effect e.g. ≦8 mg, ≦5 mg, ≦3 mg, ≦2.5 mg, etc. A minimum amount of squalene of 0.5 mg per dose is preferred (e.g. see ref. 73). Examples of amounts per dose include 5.3 mg, 4.9 mg, 2.7 mg, 2.4 mg, etc.

Methods of Treatment, and Administration of the Vaccine

Compositions of the invention are suitable for administration to human patients, and the invention provides a method of raising an immune response in a patient, comprising the step of administering a vaccine of the invention to the patient.

The invention also provides a composition of the invention for use as a medicament.

The invention also provides the use of antigens from at least two strains of influenza virus in the manufacture of a medicament for raising an immune response in a patient, where said strains include a H3N2 strain of influenza A virus, and wherein the medicament has the characteristics described above e.g. the concentration of A/H3N2 hemagglutinin is greater than 16 μg per human dose, the concentration of A/H3N2 hemagglutinin is greater than 32 μg/mL, the weight ratio of H3N2:H1N1 hemagglutinin is greater than 1. the weight ratio of H3N2:B hemagglutinin is greater than 1, etc.

These methods and uses will generally be used to generate an antibody response, preferably a protective antibody response. Methods for assessing antibody responses, neutralising capability and protection after influenza virus vaccination are well known in the art. Human studies have shown that antibody titers against hemagglutinin of human influenza virus are correlated with protection (a serum sample hemagglutination-inhibition titer of about 30-40 gives around 50% protection from infection by a homologous virus) [83]. Antibody responses are typically measured by hemagglutination inhibition, by microneutralisation, by single radial immunodiffusion (SRID), and/or by single radial hemolysis (SRH). These assay techniques are well known in the art.

Compositions of the invention can be administered in various ways. The most preferred immunisation route is by intramuscular injection (e.g. into the arm or leg) or by intradermal injection [84,85], but other available routes include subcutaneous injection, intranasal [86-88], oral [89], buccal, sublingual, transcutaneous, transdermal [90], etc.

Vaccines prepared according to the invention may be used to treat both children and adults. Influenza vaccines are currently recommended for use in pediatric and adult immunisation, from the age of 6 months. Thus the patient may be less than 1 year old, 1-5 years old, 5-15 years old, 15-55 years old, or at least 55 years old. Preferred patients for receiving the vaccines are the elderly (e.g. ≦50 years old, ≦60 years old, and preferably ≦65 years), the young (e.g. ≦5 years old), hospitalised patients, healthcare workers, armed service and military personnel, pregnant women, the chronically ill, immunodeficient patients, patients who have taken an antiviral compound (e.g. an oseltamivir or zanamivir compound; see below) in the 7 days prior to receiving the vaccine, people with egg allergies and people travelling abroad. The vaccines are not suitable solely for these groups, however, and may be used more generally in a population.

Preferred compositions of the invention satisfy 1, 2 or 3 of the CPMP criteria for efficacy. In adults (18-60 years), these criteria are: (1) ≧70% seroprotection; (2) ≧40% seroconversion; and/or (3) a GMT increase of ≧2.5-fold. In elderly (≧60 years), these criteria are: (1) ≧60% seroprotection; (2) ≧30% seroconversion; and/or (3) a GMT increase of ≧2-fold. These criteria are based on open label studies with at least 50 patients.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naïve patients e.g. for people who have never received an influenza vaccine before, or for vaccines including a new HA subtype.

Multiple doses will typically be administered at least 1 week apart (e.g. about 2 weeks, about 3 weeks, about 4 weeks, about 6 weeks, about 8 weeks, about 12 weeks, about 16 weeks, etc.).

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, a pneumococcal conjugate vaccine, etc. Administration at substantially the same time as a pneumococcal vaccine and/or a meningococcal vaccine is particularly useful in elderly patients.

Similarly, vaccines of the invention may be administered to patients at substantially the same time as (e.g. during the same medical consultation or visit to a healthcare professional) an antiviral compound, and in particular an antiviral compound active against influenza virus (e.g. oseltamivir and/or zanamivir). These antivirals include neuraminidase inhibitors, such as a (3R,4R,5S)-4-acetylamino-5-amino-3(1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g. the ethyl esters) and salts thereof (e.g. the phosphate salts). A preferred antiviral is (3R,4R,5S)-4-acetylamino-5-amino-3(1-ethylpropoxy)-1-cyclohexene-1-carboxylic acid, ethyl ester, phosphate (1:1), also known as oseltamivir phosphate (TAMIFLU™).

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a cell substrate is used for reassortment or reverse genetics procedures, or for viral growth, it is preferably one that has been approved for use in human vaccine production e.g. as in Ph Eur general chapter 5.2.3.

Modes for Carrying Out the Invention

450 patients (male & female, ≧65 years old) are split into 10 groups. Each of the groups receives a trivalent influenza vaccine (A/H1N1, A/H3N2, B). Five groups receive a standard antigen dose (3×15 μg for intramuscular, 3×6 μg for intradermal) whereas the other five groups receive a vaccine with a double dose of H3N2.

Eight of the groups (A to H) receive vaccine by intramuscular injection (0.5 mL), with or without a squalene-in-water emulsion. The vaccine is supplied in pre-filled syringes. Three different adjuvant quantities (achieved by dilution) are tested.

The other two groups (ID1 and ID2) receive unadjuvanted vaccine by the intradermal route (0.2 mL).

The influenza strains were as recommended for the influenza season 2008-2009 in the Northern Hemisphere: A/Brisbane/59/2007-like, A/Brisbane/10/2007-like virus, and B/Florida/4/2006-like.

Subjects receive a single dose and influenza-specific immune responses are assessed by analyzing blood collected at baseline (prior to vaccination), at 7 days (Day 8), and at 21 days (Day 22) after vaccination. Serum samples are assessed by strain-specific hemagglutination inhibition (HI) assays against influenza strains A/H1N1, A/H3N2 and B. Cell-mediated immunity assays are also performed.

Thus the ten groups are as follows:

| Group | H1N1 (μg) | H3N2 (μg) | B (μg) | Squalene (mg) | Dose (mL) |
|---|---|---|---|---|---|
| A | 15 | 15 | 15 | — | 0.5 |
| B | 15 | 30 | 15 | — | 0.5 |
| C | 15 | 15 | 15 | 2.44 | 0.5 |
| D | 15 | 30 | 15 | 2.44 | 0.5 |
| E | 15 | 15 | 15 | 4.88 | 0.5 |
| F | 15 | 30 | 15 | 4.88 | 0.5 |
| G | 15 | 15 | 15 | 9.75 | 0.5 |
| H | 15 | 30 | 15 | 9.75 | 0.5 |
| ID1 | 6 | 6 | 6 | — | 0.2 |
| ID2 | 6 | 12 | 6 | — | 0.2 |

All tested vaccines meet the CHMP criteria for all 3 strains. Focusing only on the immune response to the H3N2 antigen, the percentage of patients with an antibody titer >1:40 is unexpectedly lower in the patient group which receives unadjuvanted vaccines with a high H3 dose (group B; 80%) compared to the group which receives the normal dose (group A; 93%). In contrast, the percentage of patients with an antibody titer >1:40 is increased from 93% (group C) to 98% (group D) when the high H3 dose is administered in combination with 2.44 mg squalene and from 96% (group G) to 100% (group H) when the high H3 dose is administered in combination with 9.75 mg squalene. A small decrease is observed in the groups which receive the higher H3 dose in combination with 4.88 mg squalene (group F, 90% vs. 90% in group E) but an increase from 88% (group ID1) to 90% (group ID2) is seen in the groups which receive unadjuvanted vaccines intradermally.

Surprisingly, therefore, the decrease in the anti-H3 immune response seen when the high H3 dose is given without an adjuvant can be reduced, or even reversed, by either administering the vaccine (i) in combination with an adjuvant, or (ii) intradermally without an adjuvant.

It will be understood that the invention has been described by way of example only and modifications may be made whilst remaining within the scope and spirit of the invention.

### REFERENCES

[1] Burrell et al. (2008) Influenza and Other Respiratory Viruses 2(3):93-98.

[2] WO02/28422.

[3] WO02/067983.

[4] WO02/074336.

[5] WO01/21151.

[6] WO02/097072.

[7] WO2005/113756.

[8] Huckriede et al. (2003) Methods Enzymol 373:74-91.

[9] Saito et al. (2004) J Med Virol 74(2):336-43.

[10] Hoffmann et al. (2002) Vaccine 20:3165-3170.

[11] Subbarao et al. (2003) Virology 305:192-200.

[12] Liu et al. (2003) Virology 314:580-590.

[13] Ozaki et al. (2004) J. Virol. 78:1851-1857.

[14] Webby et al. (2004) Lancet 363:1099-1103.

[15] WO00/60050.

[16] WO01/04333.

[17] U.S. Pat. No. 6,649,372.

[18] Neumann et al. (2005) Proc Natl Acad Sci USA 102:16825-9.

[19] WO2006/067211.

[20] WO01/83794.

[21] Hoffmann et al. (2000) Virology 267(2):310-7.

[22] Herlocher et al. (2004) J Infect Dis 190(9):1627-30.

[23] Le et al. (2005) Nature 437(7062):1108.

[24] Gambaryan & Matrosovich (1992) J Virol Methods 39(1-2): 111-23.

[25] Mastrosovich et al. (1999) J Virol 73: 1146-55.

[26] Stevens et al. (2006) J Mol Biol 355:1143-55.

[27] Couceiro & Baum (1994) Mem Inst Oswaldo Cruz 89(4):587-91.

[28] Treanor et al. (1996) J Infect Dis 173:1467-70.

[29] Keitel et al. (1996) Clin Diagn Lab Immunol 3:507-10.

[30] WO 2008/068631.

[31] Rota et al. (1992) J Gen Virol 73:2737-42.

[32] GenBank sequence GI:325176.

[33] Kistner et al. (1998) Vaccine 16:960-8.

[34] Kistner et al. (1999) Dev Biol Stand 98:101-1 10.

[35] Bruhl et al. (2000) Vaccine 19:1149-58.

[36] Pau et al. (2001) Vaccine 19:2716-21.

[37] http://www.atcc.org/[38]

[38] http://locus.unidnledu/[39]

[39] WO03/076601.

[40] WO2005/042728.

[41] WO03/043415.

[42] WO01/85938.

[43] WO2006/108846.

[44] WO97/37000.

[45] Brands et al. (1999) Dev Biol Stand 98:93-100.

[46] Halperin et al. (2002) Vaccine 20:1240-7.

[47] Tree et al. (2001) Vaccine 19:3444-50.

[48] EP-A-1260581 (WO01/64846).

[49] WO2006/071563.

[50] WO2005/113758.

[51] WO97/37001.

[52] WO2006/027698.

[53] EP-B-0870508.

[54] U.S. Pat. No. 5,948,410.

[55] Lundblad (2001) Biotechnology and Applied Biochemistry 34:195-197.

[56] Guidance for Industry: Bioanalytical Method Validation. U.S. Department of Health and Human Services Food and Drug Administration Center for Drug Evaluation and Research (CDER) Center for Veterinary Medicine (CVM). May 2001.

[57] Ji et al. (2002) Biotechniques. 32:1162-7.

[58] Briggs (1991) J Parenter Sci Technol. 45:7-12.

[59] Lahijani et al. (1998) Hum Gene Ther. 9:1173-80.

[60] Lokteff et al. (2001) Biologicals. 29:123-32.

[61] Gennaro (2000) Remington: The Science and Practice of Pharmacy. 20th edition, ISBN: 0683306472.

[62] Banzhoff (2000) Immunology Letters 71:91-96.

[63] O'Hagan (2007) Expert Rev Vaccines. 6(5):699-710.

[64] Bernstein et al. (2008) J Infect Dis. 197(5):667-75.

[65] Stephenson et al. (2005) J Infect Dis. 191(8):1210-5.

[66] Han et al. (2005) Impact of Vitamin E on Immune Function and Infectious Diseases in the Aged at Nutrition, Immune functions and Health EuroConference, Paris, 9-10 June 2005.

[67] U.S. Pat. No. 6,630,161.

[68] WO90/14837.

[69] Podda & Del Giudice (2003) Expert Rev Vaccines 2:197-203.

[70] Podda (2001) Vaccine 19: 2673-2680.

[71] Vaccine Design: The Subunit and Adjuvant Approach (eds. Powell & Newman) Plenum Press 1995 (ISBN 0-306-44867-X).

[72] Vaccine Adjuvants: Preparation Methods and Research Protocols (Volume 42 of Methods in Molecular Medicine series). ISBN: 1-59259-083-7. Ed. O'Hagan.

[73] WO2008/043774.

[74] Allison & Byars (1992) Res Immunol 143:519-25.

[75] Hariharan et al. (1995) Cancer Res 55:3486-9.

[76] US-2007/0014805.

[77] US-2007/0191314.

[78] Suli et al. (2004) Vaccine 22(25-26):3464-9.

[79] WO95/11700.

[80] U.S. Pat. No. 6,080,725.

[81] WO2005/097181.

[82] WO2006/113373.

[83] Potter & Oxford (1979) Br Med Bull 35: 69-75.

[84] Halperin et al. (1979) Am J Public Health 69:1247-50.

[85] Herbert et al. (1979) J Infect Dis 140:234-8.

[86] Greenbaum et al. (2004) Vaccine 22:2566-77.

[87] Zurbriggen et al. (2003) Expert Rev Vaccines 2:295-304.

[88] Piascik (2003) J Am Pharm Assoc (Wash DC). 43:728-30.

[89] Mann el al. (2004) Vaccine 22:2425-9.

[90] Chen et al. (2003) Vaccine 21:2830-6.

1. An inactivated influenza virus vaccine including an A/H1N1 hemagglutinin and a A/H3N2 hemagglutinin, wherein the weight ratio of H3N2:H1N1 hemagglutinin is greater than 1 and wherein the vaccine includes an adjuvant.

2. An inactivated influenza virus vaccine including an A/H3N2 hemagglutinin and a B hemagglutinin, wherein the weight ratio of H3N2:B hemagglutinin is greater than 1, and wherein the vaccine includes an adjuvant.

3. The vaccine of claim 1, comprising hemagglutinins from an A/H1N1 influenza virus strain, an A/H3N2 influenza virus strain, and an influenza virus B strain, wherein (i) the weight ratio of H3N2:H1N1 hemagglutinin is greater than 1 and (ii) the weight ratio of H3N2:B hemagglutinin is greater than 1.

4. An inactivated influenza virus vaccine including A/H3N2 hemagglutinin, wherein the concentration of A/H3N2 hemagglutinin is greater than 32 μg/mL, and wherein the vaccine includes an adjuvant.

5. The vaccine of claim 1, wherein the vaccine is a split virion vaccine or a purified surface antigen vaccine.

6. The vaccine of claim 1, wherein the concentration of A/H3N2 hemagglutinin is about 60 μg/ml.

7. The vaccine of any preceding claim 1, wherein the concentration of hemagglutinin for influenza virus strains other than A/H3N2 are in the range 0.2-30 μg/ml per strain.

8. The vaccine of claim 1, comprising hemagglutinin from multiple influenza virus strains, and wherein the amount of A/H3N2 hemagglutinin is higher than the average amount of hemagglutinin per strain.

9. The vaccine of claim 1, wherein the amount of A/H3N2 hemagglutinin is equal to the combined amount of hemagglutinin from non-A/H3N2 influenza virus strains.

10. The vaccine of claim 1, wherein the vaccine is a trivalent inactivated vaccine containing hemagglutinin from a H1N1 influenza virus A strain, a H3N2 influenza virus A strain, and an influenza B virus strain, wherein the hemagglutinin weight ratio is 1:2:1 (A/H1N1: A/H3N2: B).

11. The vaccine of claim 1, comprising an oil-in-water emulsion adjuvant with submicron oil droplets.

12. The vaccine of claim 11, wherein the emulsion is a squalene-containing emulsion.

13. The vaccine of claim 12, wherein the emulsion comprises squalene, polysorbate 80, and sorbitan trioleate.

14. The vaccine of claim 12, wherein the emulsion comprises squalene, DL-α-tocopherol, and polysorbate 80.

15. The vaccine of claim 12, wherein the emulsion comprises squalene, an aqueous solvent, a polyoxyethylene alkyl ether hydrophilic nonionic surfactant and a hydrophobic nonionic surfactant.

16. The vaccine of claim 12, with between 18 mg/mL and 22 mg/mL squalene.

17. The vaccine of claim 12, with between 2 mg and 8 mg squalene per human dose.

18. A method of raising an immune response in a patient, comprising the step of administering the vaccine of claim 1 to the patient.

19. An intradermal inactivated influenza vaccine including:
(a) an A/H1N1 hemagglutinin and a A/H3N2 hemagglutinin, wherein the weight ratio of H3N2:H1N1 hemagglutinin is greater than 1;
(b) an A/H1N1 hemagglutinin and a B hemagglutinin, wherein the weight ratio of H3N2:B hemagglutinin is greater than 1; or
(c) A/H3N2 hemagglutinin, wherein the concentration of A/H3N2 hemagglutinin is greater than 32 μg/mL.

20. The use of a vaccine according to claim 19 for intradermal administration.
